(19) United States
(12) Patent Application Publication
(10) Pub. No.: US 2026/0295212 A1
(43) Pub. Date: Oct. 1, 2026

(54) PACKAGED LUBRICATED READY-TO-USE INTERMITTENT URINARY CATHETER

(71) Applicant: ConvaTec Inc., Bridgewater, NJ (US)

(72) Inventor: Timothy Palmer, Lexington, MA (US)

(21) Appl. No.: 19/013,506

(22) PCT Filed: Jun. 26, 2023
(86) PCT No.: PCT/US2023/069095

§ 371 Date: Jan. 8, 2025

Related U.S. Application Data
(60) Provisional application No. 63/357,761, filed on Jul. 1, 2022.

(51) Int. Cl. A61M 25/00

(52) U.S. Cl. CPC A61M 25/002; A61M 25/0041; A61M 25/007; A61M 2205/0222; A61M 2207/00; A61M 2209/06

(57) ABSTRACT

A packaged ready-to-use intermittent urinary catheter includes first and second layers of film, the urinary catheter being vacuum sealed in a retention chamber between the first and second layers of film, a pair of spaced-apart collars slidingly positioned on the catheter and in sealing contact with the first and second layers of film, and lubricant in the retention chamber surrounding the catheter tube and between the spaced-apart collars. The packaging for the catheter is formed by slidingly positioning a pair of spaced-apart collars on the catheter, applying lubricant onto the catheter between the collars, placing the catheter within a depression formed in a first or base film, placing a second or cover film sealed over the base film to form a retention chamber, and a pulling a vacuum within the retention chamber.

### FIELD

The present application relates to packaged intermittent urinary catheters and, in particular, to a packaged catheter having an improved lubricant gel configuration.

### BACKGROUND

Intermittent catheters are typically used by patients suffering from urinary incontinence or by individuals unable to enjoy voluntary urination. In our highly mobile culture, the ability to have the freedom to leave home for the day or longer is an important part of life. To accommodate this need single use, pre-lubricated urinary catheters have been developed to allow patients to perform self-catheterization. An individual requiring catheterization will typically utilize several catheters every day. This results in the usage of a large number of intermittent urinary catheters over time, driving a demand for inexpensive catheters without sacrificing safety and comfort.

A particular complication encountered with the production and transport of inexpensive, single use, pre-lubricated, ready for use intermittent urinary catheters for self-catheterization is constraining the lubricant to the insertion portion of the packaged catheter so as to prevent lubrication of the fixture end portion of the catheter which must be gripped during insertion and removal of the catheter.

Accordingly, a particular need exists for an inexpensive method for packaging an intermittent urinary catheter that constrains the lubricant to contact with the insertion portion, and prevents migration of the lubricant within the package into contact with the fixture end of the catheter under normal storage and use conditions.

### SUMMARY OF THE INVENTION

Embodiments of the present invention provide an efficiently packaged, ready to use intermittent urinary catheter. The packaged catheter includes, and except for appropriate labeling and marking preferably only includes, (i) an intermittent urinary catheter, (ii) packaging formed from first and second layers of film, (iii) a pair of spaced-apart collars slidingly positioned on the catheter, and (iv) a lubricant. The catheter has a longitudinal axial length, an insertion end, a funnel end and a top view profile, and is hermetically packaged with the collars between the first and second layers of film within a retention chamber formed between the films. The retention chamber has a top view profile conforming to the top view profile of the catheter and retains a supply of lubricant within a lubricant retaining compartment formed between the two collars in the retention chamber.

A first packaged lubricated ready-to-use intermittent urinary catheter, comprises a urinary catheter having an elongated flexible catheter tube extending from a proximal outlet fixture to a rounded distal tip on a distal end and one or more discharge openings proximate the distal tip. Packaging formed from first and second layers of film is sealingly attached at border regions such that a distal border region seals distally beyond the distal tip, and a proximal border region seals proximally beyond the proximal outlet fixture and along lateral edges therebetween. The urinary catheter is vacuum sealed with a negative pressure in a retention chamber between the first and second layers of film. Spaced-apart proximal and distal packaging collars are slidingly positioned on the catheter and in sealing contact with the first and second layers of film, the proximal packaging collar being spaced distally from the proximal outlet fixture and the distal packaging collar being spaced proximally from the distal tip. A lubricant positioned in the retention chamber surrounds the catheter tube only between the packaging collars such that a lubricated length of the catheter exists between the packaging collars, and wherein the packaging collars inhibit migration of the lubricant beyond the lubricated length of the catheter.

A second packaged lubricated ready-to-use intermittent urinary catheter, comprises a urinary catheter having an elongated flexible catheter tube extending from a proximal outlet fixture to a rounded distal tip on a distal end and one or more discharge openings proximate the distal tip. Packaging is formed from first and second layers of film sealingly attached at border regions such that a distal border region seals distally beyond the distal tip, and a proximal border region seals proximally beyond the proximal outlet fixture and along lateral edges therebetween. Spaced-apart proximal and distal packaging foam collars are slidingly positioned on the catheter and in sealing contact with the first and second layers of film, the proximal packaging collar being spaced distally from the proximal outlet fixture and the distal packaging collar being spaced proximally from the distal tip. Finally, lubricant is positioned in the retention chamber surrounding the catheter tube only between the packaging collars such that a lubricated length of the catheter exists between the packaging collars, wherein the packaging collars inhibit migration of the lubricant beyond the lubricated length of the catheter.

In the packaged catheters, inner lumens of the packaging collars may be sized the same as or slightly smaller than an outer diameter of the catheter tube. The packaging collars are desirably formed of a polymeric foam. The retention chamber may be formed by a molded cavity in the first layer of film shaped to conform to an external shape of the catheter, while the second layer of film is flat. In one embodiment, the retention chamber is molded to have a constant width portion sized to conform to the catheter tube and interrupted by a proximal enlarged pocket for receiving the proximal packaging collar. The constant width portion may also be interrupted by a distal enlarged pocket for receiving the distal packaging collar. The proximal enlarged pocket may be cylindrical and the proximal packaging collar is cylindrical and sized larger than the proximal enlarged pocket.

The first and second layers of film at the proximal end of the packaging of the packaged catheter may be separated to form tabs configured to facilitate peeling the layers of film apart to access the urinary catheter, and the packaging further includes a lateral strip of extra bonding located between the proximal outlet fixture and the proximal packaging collar to prevent peeling the layers of film apart past the lateral strip of extra bonding.

In the packaged catheters described herein, the distal packaging collar may be positioned at least 1-2 cm from the distal tip of the catheter tube. Further, the urinary catheter may have a length of between 15-42 cm, with the packaging collars spaced apart between 5-27 cm.

A method of packaging an intermittent urinary catheter, comprises:
a. providing a urinary catheter having an elongated flexible catheter tube extending from a proximal outlet fixture to a rounded distal tip on a distal end and one or more discharge openings proximate the distal tip;
b. positioning proximal and distal packaging collars on the catheter at a spaced-apart distance, the proximal packaging collar being spaced distally from the proximal outlet fixture and the distal packaging collar being spaced proximally from the distal tip;
c. providing packaging including first and second layers of film;
d. placing the urinary catheter and packaging collars on the first layer of film and covering the urinary catheter with the second layer of film;
e. depositing lubricant along the catheter tube only between the packaging collars such that a lubricated length of the catheter exists between the packaging collars; and
f. creating a negative pressure in a retention chamber between the first and second layers of film and sealingly attaching the first and second layers of film at border regions such that a distal border region seals distally beyond the distal tip, and a proximal border region seals proximally beyond the proximal outlet fixture and along lateral edges therebetween, the urinary catheter and packaging collars thus being vacuum sealed in the retention chamber, wherein the packaging collars inhibit migration of the lubricant beyond the lubricated length of the catheter.

Another aspect is a method of efficiently packaging a precision lubricated ready to use intermittent urinary catheter having an insertion end, an insertion end length, a fixture end, and a fixture end length. The method includes the steps of (a) obtaining a base film having an elongated depression formed between a first end and a second end, wherein the depression is configured to retain the intermittent urinary catheter and a pair of spaced-apart collars thereon with the insertion end proximate the first end and the fixture end proximate the second end, (b) injecting a limited amount of lubricant on the catheter between the two collars, (c) placing the intermittent urinary catheter and collars into the depression, with the insertion end of the intermittent urinary catheter proximate the first end and the fixture end of the intermittent urinary catheter proximate the second end, (d) sealing a cover film to the base film with an unsealed opening proximate the second end of the pocket so as to form an enclosed catheter containing retention chamber accessible through the opening, (e) drawing a vacuum on the enclosed catheter containing retention chamber through the opening sufficient to compress the retention chamber and cause lubricant to flow within the retention chamber between the collars without reaching and coating the fixture end length of the catheter, and thereafter (f) sealing the opening so as to form a hermetically packaged intermittent urinary catheter having a lubricated insertion end length and a lubricant-free fixture end length.

### BRIEF DESCRIPTION OF THE DRAWINGS

FIG. 1 is a perspective view of a conventional intermittent urinary catheter shown with a pair of packaging collars of the present application thereon;

FIG. 2 is a perspective view of a packaged intermittent urinary catheter of the present application, with the catheter between first and second film layers, and FIG. 2A is an enlarged view of an alternative proximal end of the packaging;

FIG. 3 is a plan view of the packaged intermittent urinary catheter of FIG. 2 showing the first and second film layers being separated during an opening procedure;

FIG. 4 is a plan view of an alternative bottom film layer and urinary catheter therein; and

FIG. 5 is a plan view of a further alternative bottom film layer and urinary catheter therein.

### DETAILED DESCRIPTION OF A PREFERRED EMBODIMENT

The present application provides a packaged ready-to-use intermittent urinary catheter includes first and second layers of film, the urinary catheter being vacuum sealed in a retention chamber between the first and second layers of film, a pair of spaced-apart collars slidingly positioned on the catheter and in sealing contact with the first and second layers of film, and lubricant in the retention chamber surrounding the catheter tube and between the spaced-apart collars.

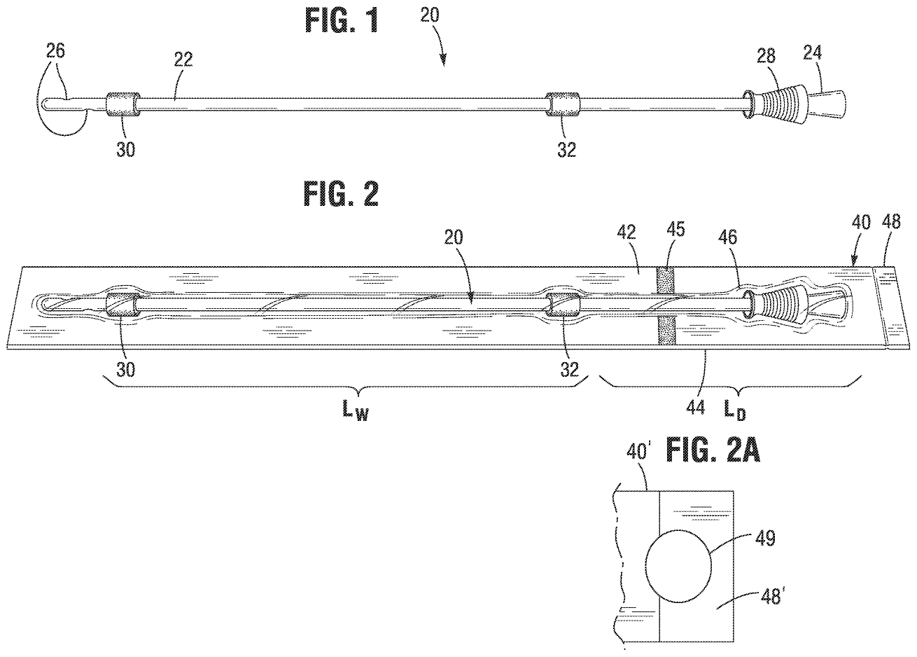

FIG. 1 is a perspective view of a conventional urinary catheter 20 modified as explained herein. The urinary catheter 20 has an elongated flexible catheter tube 22 extending from a proximal outlet fixture 24 to a rounded tip on a distal end, and one or more discharge openings 26 proximate the tip. The outlet fixture 24 is shown as a flexible (e.g., elastomeric) funnel. The discharge openings 26 are provided with rounded and or polished rims, such that the entry of urethral catheter tube 22 into the urethral tract of a patient is as comfortable as possible for the patient. A flexible (e.g., elastomeric) grip device 28 that surrounds and may be slide along the catheter tube 22 is provided to facilitate usage of the catheter 20.

As utilized herein, including the claims, the term “fixture” means and refers to the well-known commercially available components commonly attached to the proximal non-insertion end of urinary catheters, including specifically but not exclusively funnels, luer locks, clamps, valves, etc.

When ready to use, the catheter 20 is removed from its sterile packaging. The user can hold the proximal outlet fixture 24 in one hand, while sliding the flexible grip device 28 along the length of the catheter towards the distal end thereof. Lubricant is already applied to the distal end from various packaging solutions or in a manual step after removing the catheter from the packaging. The user carefully inserts the rounded distal tip of the catheter into the urethra opening, and begins the process of advancing the catheter tube 22. While holding the proximal outlet fixture 24 over a toilet or other waste reservoir, the user manipulates the grip device 28 back and forth along the catheter tube 22 until the discharge openings 26 reach the bladder, at which point urine begins to flow in a proximal direction through the catheter tube 22. Once the bladder is empty, the user reverses the process by manipulating the catheter tube 22 out of the urethra using (i.e., squeezing and pulling with) the grip device 28, as well as pulling on the proximal outlet fixture 24.

As mentioned, there are a number of packaging solutions to ensure that the distal tip of the catheter tube 22 has sufficient lubricant gel or other lubrication (e.g., a hydrophilic coating activated by water). Problems have arisen from fluid lubrication within the catheter packaging migrating to the proximal outlet fixture 24 or to the grip device 28, which can hinder the insertion process by making these “handles” somewhat slippery.

In response, FIG. 1 shows a pair of packaging collars 30, 32 mounted around the catheter tube 22, and FIG. 2 is a perspective view of the urinary catheter 20 with the collars 30, 32 held in a sterile package 40 of the present application. In the illustrated embodiment, the packaging collars 30, 32 are tubular compressible members, such as polymer foam, which are easily slid onto the catheter tube 22 in interference fits. More specifically, inner lumens of the collars 30, 32 are sized the same as or slightly smaller than the outer diameter of the catheter tube 22.

The sterile package 40 comprises a first or top film layer 42 adhered to a second or bottom film layer 44, with the catheter 20 and collars 30, 32 sandwiched therebetween. More particularly, the catheter 20 and collars 30, 32 thereon are positioned within an elongated retention chamber 46 defined between the film layers 42, 44. The film layers 42, 44 are adhered or otherwise secured together in all of the border areas surrounding the retention chamber 46, but the chamber has molded cavities or pockets shaped to conform to the catheter 20 so as to constrain the catheter therein without undue force or adhesive.

In the illustrated embodiment, the first and second film layers 42, 44 are elongated rectangles having a length and width sufficient to extend longitudinally beyond the proximal and distal ends of the catheter, and laterally on either side thereof. Intermittent urinary catheters 20 typically range in length between 15-42 cm, with catheters for females being shorter, and have a maximum width at the proximal outlet end of around 1.8 cm (grip device for male catheters). The packaging 40 thus extends outward from the retention chamber 46 by between about 2.0-2.5 cm in border areas to ensure the package will remain sterile without becoming delaminated in shipping or storage. This results in a maximum total package width of 4.0 to 5.0 cm.

A lateral strip 45 of extra bonding between the first and second film layers 42, 44 may be provided. The extra bonding may be from heat sealing or welding, or by using a different, more secure adhesive than elsewhere. As will be seen, the adhesive used around the majority of the edges of the packaging 40 is that which maintains a sterile bond but which can be pulled apart manually. To facilitate separating the first and second film layers 42, 44, proximal end tabs 48 are left without adhesive so as to form two separate tabs that can be gripped to effectuate manual separation of the layers. However, the lateral strip 45 of extra bonding prevents complete separation of the layers 42, 44, described below.

FIG. 2A shows a proximal end of an alternative package 40′ with longer separated tabs 48′, up to 3 cm or more. In addition, finger openings 49 through both tabs 48′ are provided to reduce the gripping power needed. That is, once separated, the user pulls on the upper tab 48′ using the finger hole 49.

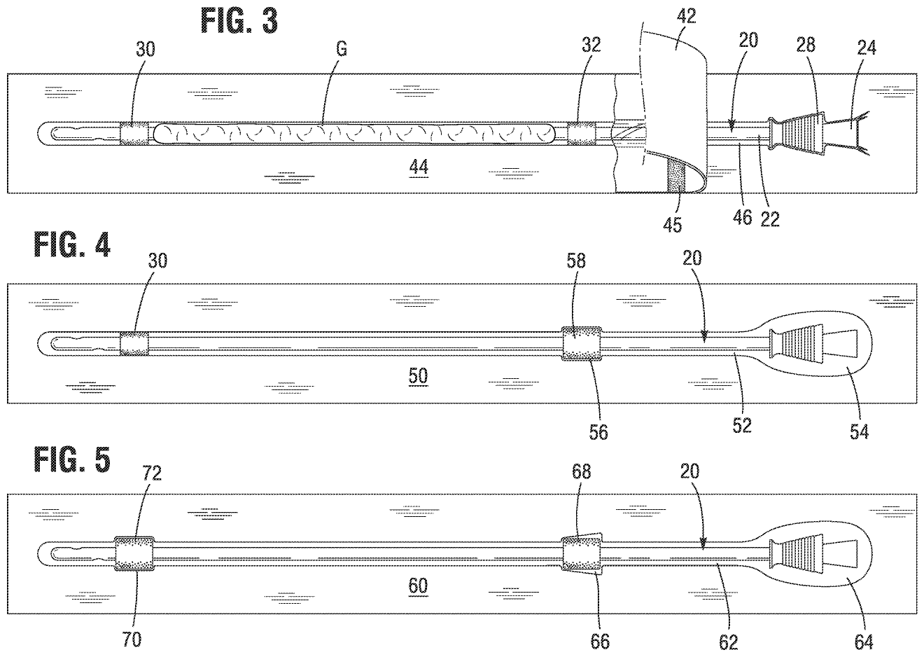

FIG. 3 is a plan view of the packaged intermittent urinary catheter of FIG. 2 showing the first and second film layers 42, 44 being separated during an opening procedure. Namely, the separated end tab 48 of the first or top layer 42 is gripped and pulled away from the end tab 48 of the second or bottom layer 44. The two layers 42, 44 separate until reaching the lateral strip 45. Because the strip 45 has extra bonding, further separation of the two layers 42, 44 is halted. At this point, the proximal end of the urinary catheter 20, including the proximal outlet fixture 24 and grip device 28 are exposed, enabling the user to pull the proximal end upward from the retention chamber and extract the catheter 20 from the package 40, while leaving behind the packaging collars 30, 32. Consequently, the configuration seen in FIG. 1 is one that occurs during assembly of the urinary catheter 20 into the sterile package 40. Once assembled, the catheter 20 may be removed from the package 40 but the collars 30, 32 remain.

The packaging collars 30, 32 are shown spaced apart along the catheter tube 22 with a quantity of lubricating gel G placed therebetween. In the illustrated embodiment, the collars 30, 32 are tubular foam elements having a diameter which is approximately the same as the lateral width of the retention chamber 46 along most of its length. That is, the retention chamber 46 extends from close to the distal end of the catheter tube 22 in a constant width until it reaches the proximal end thereof, where a larger pocket is formed to receive and conform to the proximal outlet fixture 24 and grip device 28. A brief description of the process of fabricating the packaging 40 is appropriate.

A first step in the process is to form the second or bottom film layer 44 from an initially planar blank of film. The retention chamber 46 is a depression molded into the planar blank in the approximate shape of the catheter 20. In the embodiment of FIGS. 2 and 3, the retention chamber 46 has a rounded distal end, a constant lateral width extending along a majority of its length, and a molded pocket at its proximal end which generally conforms to the contours of the proximal outlet fixture 24 and grip device 28. In this embodiment, both the outlet fixture 24 and the grip device 28 have generally conical, funnel-shapes, though other configurations are possible.

The urinary catheter 20 is then placed within the molded depression of the retention chamber 46, and the lubricating gel G placed (e.g., injected) in the chamber between the collars 30, 32. As mentioned, the tubular collars 30, 32 both have a diameter which approximately equals the lateral width of the retention chamber 46. Subsequently, the top or first film layer 42 is placed over the bottom layer 44 and adhered thereto. In one embodiment, adhesive is applied along the outer borders of the bottom layer 44 outside of the retention chamber 46. The top layer 42 is desirably a planar blank with no adhesive. Alternatively, the border regions of the top and bottom layers 42, 44 may be welded or heat sealed together.

The second or top film layer 42 is then sealed to the base film layer 44 with an unsealed opening proximate one end of the retention chamber 46 so as to form an enclosed catheter containing retention chamber accessible through the opening. The unsealed opening could be anywhere on the package 40 and is typically one of the long sides due to equipment configuration. A vacuum is then drawn on the enclosed catheter containing retention chamber 46 through the opening sufficient to compress the retention chamber and cause lubricant to flow within the retention chamber between the collars 30, 32 without reaching and coating the fixture end length of the catheter. Thereafter, the opening is sealed so as to form a hermetically packaged intermittent urinary catheter retention chamber 46 having a lubricated or wet insertion end length Lw and a lubricant-free or dry fixture end length LD, as shown in FIG. 2. This helps conform the two sandwiched layers 42, 44 together and collapse the retention chamber 46 around the catheter 20 (see wrinkled proximal end in FIG. 2). Finally, the extra bonding along the lateral strip 45 is created, such as by heat sealing, or a stronger adhesive is pre-applied to reduce steps.

The result is a retention chamber 46 which hugs the exterior of the catheter 20, and in particular contacts and seals against both of the packaging collars 30, 32. Consequently, the quantity of lubricating gel G placed between the collars 30, 32 is sealed within that region and prevented from migrating by contact between the collars and the sandwiched layers 42, 44. Namely, no gel migrates proximally past collar 32 to reach the outlet fixture 24 and grip device 28, and none migrates distally beyond distal collar 30, which might enter the drainage eyelets 26 and then travel up towards the funnel and also contaminate the outlet fixture 24. When the user peels back the top layer 42 to remove the catheter 20, none of the gel has migrated to the proximal outlet fixture 24 or grip device 28 they are dry.

The user can then easily withdraw the rest of the catheter tube 22 out of the packaging 40, and the packaging collars 30, 32 remain in place by an interference/friction fit. A layer of gel G coats the exterior of the distal portion of the catheter tube 22 as it is removed from the packaging, thus facilitating insertion into the urethra. The packaging collars 30, 32 also ensure that a controlled amount of gel is applied to the catheter upon withdrawal and prevent any excess lubricant from exiting the package once the catheter has been removed to keep the environment clean.

It should be noted that though a vacuum-sealed package is described and works well, the package could also be assembled without a negative pressure within the retention chamber. A “negative pressure” in this context means less than atmospheric pressure, preferably down to pressures conventionally used in vacuum packaging. That is, various manufacturing methods are known to enable two film layers be compressed and sealed around internal objects. For instance, the catheter/collars may be positioned between two film layers and the layers compressed against the catheter using a vacuum-type of flexible press. That is, no negative pressure exists inside the package, but vacuum within an outer press bag is used to conform the film layers around the inner catheter and collars. Similarly, a soft press that conforms the film layers around the inner catheter and collars merely using compressive clamping (i.e., no vacuum) is also possible. Though a negative pressure within the retention chamber is considered to provide a good seal to prevent migration of the lubricant from the length between the collars, adequate seals could also be established with the proper manufacturing techniques, such as ensuring adhesives or bonding between the film layers as close as possible around the internal objects coupled with a conventional or vacuum press.

As mentioned, urinary catheters 20 typically range in length between 15-42 cm, and the collars 30, 32 may be placed in a number of locations along the catheter tube 22 which allows for a suitable amount of gel G to be injected. For instance, the collars 30, 32 may be spaced apart by between 5-27 cm, depending on whether the catheter is a shorter length for females or longer for males. The distal collar 30 is positioned at least 1-2 cm from the distal tip of the catheter tube 22.

FIG. 4 is a plan view of an alternative bottom film layer 50 and urinary catheter 20 therein prior to assembly with a top film layer. As before, a retention chamber 52 including an enlarged proximal end 54 is molded into the bottom film layer 50. In this embodiment, an enlarged cylindrical secondary pocket 56 is molded as part of the retention chamber 52 for receiving a proximal packaging collar 58, which is tubular and has a larger diameter than a distal packaging collar 30. The fabrication of the catheter packaging proceeds as described above, with the addition of a flat top film layer and vacuum pulled to shrink the retention chamber 52 around the exterior of the catheter 20. Because of the larger size of the proximal packaging collar 58 and pre-formed pocket 56, there is less chance of the proximal collar 58 being pulled from within the sandwiched packaging layers upon withdrawal. Furthermore, it is believed that a larger packaging collar 58 in combination with a pre-formed retention pocket 56 provides a better seal against migration of the lubricant gel G from within the space between the collars.

FIG. 5 is a plan view of a further alternative bottom film layer 60 and urinary catheter 20 therein. Again, the film layer 60 has a retention chamber 62 molded therein, which is provided with an enlarged proximal end 64 and a secondary pocket 66 that conforms around a proximal packaging collar 68. In this embodiment, the proximal packaging collar 68 is once again tubular and has a larger diameter than the collars 30, 32 of FIG. 3. Rather than also being cylindrical, the secondary pocket 66 has a frustoconical configuration which may improve the ability to seal around the collar 68 and prevent gel migration. Furthermore, the retention chamber 62 defines a cylindrical tertiary pocket 70 positioned and sized to conform to an enlarged distal packaging collar 72. Once again, enlarging the distal collar 72 and providing a pre-formed pocket 70 is believed to enhance the seal therebetween to prevent gel migration.

In one embodiment, the packaging collars are made of polyethylene foam and have tubular configurations for more uniform sealing within the film layers. The foam could be either open cell (Polyurethane or other) or closed cell (Polyethylene or other), and in various durometers. Of course, other compressible materials such as silicone or the like could be used.

The lubricant gel G is preferably a silicone-based gel as opposed to a silicone oil. The gel has more body to it and also has additives to give a bit of cushion to the gel. One promising silicone gel G has a lower viscosity (10,000-25,000 cp) than previous lubricants used. Many lubricants currently used are water-based gels made primarily from glycerin and water.

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

It is claimed:

1. A packaged lubricated ready-to-use intermittent urinary catheter, comprising:
a urinary catheter having an elongated flexible catheter tube extending from a proximal outlet fixture to a rounded distal tip on a distal end and one or more discharge openings proximate the distal tip;
packaging formed from first and second layers of film sealingly attached at border regions such that a distal border region seals distally beyond the distal tip, and a proximal border region seals proximally beyond the proximal outlet fixture and along lateral edges therebetween, the urinary catheter being vacuum sealed with a negative pressure in a retention chamber between the first and second layers of film;
spaced-apart proximal and distal packaging collars slidingly positioned on the catheter and in sealing contact with the first and second layers of film, the proximal packaging collar being spaced distally from the proximal outlet fixture and the distal packaging collar being spaced proximally from the distal tip; and
lubricant in the retention chamber surrounding the catheter tube and positioned only between the packaging collars such that a lubricated length of the catheter exists between the packaging collars, wherein the packaging collars inhibit migration of the lubricant beyond the lubricated length of the catheter.

2. The packaged catheter of claim 1, wherein inner lumens of the packaging collars are sized the same as or slightly smaller than an outer diameter of the catheter tube.

3. The packaged catheter of either of claims 1 or 2, wherein the packaging collars are formed of a polymeric foam.

4. The packaged catheter of any preceding claim, wherein the retention chamber is formed by a molded cavity in the first layer of film shaped to conform to an external shape of the catheter, while the second layer of film is flat.

5. The packaged catheter of claim 4, wherein the retention chamber is molded to have a constant width portion sized to conform to the catheter tube and interrupted by a proximal enlarged pocket for receiving the proximal packaging collar.

6. The packaged catheter of claim 5, wherein the constant width portion is also interrupted by a distal enlarged pocket for receiving the distal packaging collar.

7. The packaged catheter of claim 5, wherein the proximal enlarged pocket is cylindrical and the proximal packaging collar is cylindrical and sized larger than the proximal enlarged pocket.

8. The packaged catheter of any preceding claim, wherein the first and second layers of film at the proximal end of the packaging are separated to form tabs configured to facilitate peeling the layers of film apart to access the urinary catheter, and the packaging further includes a lateral strip of extra bonding located between the proximal outlet fixture and the proximal packaging collar to prevent peeling the layers of film apart past the lateral strip of extra bonding.

9. The packaged catheter of any preceding claim, wherein the distal packaging collar is positioned at least 1-2 cm from the distal tip of the catheter tube.

10. The packaged catheter of any preceding claim, wherein the urinary catheter has a length of between 15-42 cm, and the packaging collars are spaced apart between 5-27 cm.

11. A method of packaging an intermittent urinary catheter, comprising:
providing a urinary catheter having an elongated flexible catheter tube extending from a proximal outlet fixture to a rounded distal tip on a distal end and one or more discharge openings proximate the distal tip;
positioning proximal and distal packaging collars on the catheter at a spaced-apart distance, the proximal packaging collar being spaced distally from the proximal outlet fixture and the distal packaging collar being spaced proximally from the distal tip;
providing packaging including first and second layers of film;
placing the urinary catheter and packaging collars on the first layer of film and covering the urinary catheter with the second layer of film;
depositing lubricant along the catheter tube only between the packaging collars such that a lubricated length of the catheter exists between the packaging collars; and
creating a negative pressure in a retention chamber between the first and second layers of film and sealingly attaching the first and second layers of film at border regions such that a distal border region seals distally beyond the distal tip, and a proximal border region seals proximally beyond the proximal outlet fixture and along lateral edges therebetween, the urinary catheter and packaging collars thus being vacuum sealed in the retention chamber, wherein the packaging collars inhibit migration of the lubricant beyond the lubricated length of the catheter.

12. The method of claim 11, wherein inner lumens of the packaging collars are sized the same as or slightly smaller than an outer diameter of the catheter tube, and the packaging collars are formed of a polymeric foam.

13. The method of any of claims 11 or 12, wherein the retention chamber is formed by a molded cavity in the first layer of film shaped to conform to an external shape of the catheter, while the second layer of film is flat.

14. The method of any of claims 11, 12 or 13, wherein the first and second layers of film at the proximal end of the packaging are separated to form tabs configured to facilitate peeling the layers of film apart to access the urinary catheter, and the packaging further includes a lateral strip of extra bonding located between the proximal outlet fixture and the proximal packaging collar to prevent peeling the layers of film apart past the lateral strip of extra bonding.

15. The method of any of claims 11, 12, 13 or 14, wherein the distal packaging collar is positioned at least 1-2 cm from the distal tip of the catheter tube, the urinary catheter has a length of between 15-42 cm, and the packaging collars are spaced apart between 5-27 cm.
